(19) United States
(12) Patent Application Publication
(10) Pub. No.: US 2006/0264954 A1
(43) Pub. Date: Nov. 23, 2006

(54) Active compression screw system and method for using the same

(76) Inventors: Thomas M. Sweeney II, Sarasota, FL (US); David T. Hawkes, Pleasant Grove, UT (US)

Correspondence Address: RADER, FISHMAN & GRAUER PLLC, 10653 SOUTH RIVER FRONT PARKWAY, SUITE 150, SOUTH JORDAN, UT, 84095, US

(21) Appl. No.: 11/399,729

(22) Filed: Apr. 6, 2006

Related U.S. Application Data
(60) Provisional application No. 60/669,498, filed on Apr. 7, 2005.

(51) Int. Cl. A61B 17/58

(52) U.S. Cl. 606/73

(57) ABSTRACT

An active compression orthopedic screw includes a first shaft member positioned at a distal end of the screw, a second shaft member positioned at a proximal end of the screw, and an elastic member having a first and a second end. According to one exemplary embodiment, the first end of the elastic member is coupled to the first shaft member and said second end of the elastic member is coupled to the second shaft member, the elastic member being configured to exert a force drawing the first and second shaft members together.

### RELATED APPLICATIONS

This application claims the benefit under 35 U.S.C. § 119(e) of U.S. Provisional Patent Application No. 60/699,498 filed Apr. 7, 2005 titled “Active Fracture Screw.” The provisional application is incorporated herein by reference in its entirety.

### FIELD

The present system and method relate to bone fixation devices. More particularly, the present system and method provide for an active compression screw system that may be used to fix soft tissue or tendons to bone or for securing two or more adjacent bone fragments or bones together.

### BACKGROUND

In the treatment of various orthopedic conditions, including the treatment of fractures, tumors, and degenerative conditions, it is often necessary to secure and stabilize segments of bone. Various devices for internal fixation of bone segments in the human or animal body are known in the art.

Bones which have been fractured, either by accident or severed by surgical procedure must be kept together for lengthy periods of time in order to permit the recalcification and bonding of the severed parts. Accordingly, adjoining parts of a severed or fractured bone are typically clamped together or attached to one another by means of a pin or a screw driven through the rejoined parts. Movement of the pertinent part of the body may then be kept at a minimum, such as by application of a cast, brace, splint, or other conventional technique, in order to promote healing and avoid mechanical stresses that may cause the bone parts to separate during bodily activity.

The surgical procedure of attaching two or more parts of a bone with a pin-like device requires an incision into the tissue surrounding the bone and the drilling of a hole through the bone parts to be joined. Due to the significant variation in bone size, configuration, and load requirements, a wide variety of bone fixation devices have been developed. In general, the current standard of care relies upon a variety of metal wires, screws, and clamps to stabilize the bone fragments during the healing process.

Some bone fixation fasteners have been developed that provide for the joining of two or more bone parts for compressive bone fixation. However, traditional bone fixation fasteners only apply a passive compression across a fracture.

### SUMMARY

According to one exemplary embodiment, an orthopedic bone fixation screw for actively compressing a plurality of bone segments includes a first shaft member positioned at a distal end of the screw, a second shaft member positioned at a proximal end of the screw, and an elastic member having a first and a second end. According to one exemplary embodiment, the first end of the elastic member is coupled to the first shaft member and said second end of the elastic member is coupled to the second shaft member, the elastic member being configured to exert a force drawing the first and second shaft members together.

### BRIEF DESCRIPTION OF THE DRAWINGS

The accompanying drawings illustrate various exemplary embodiments of the present system and method and are a part of the specification. Together with the following description, the drawings demonstrate and explain the principles of the present system and method. The illustrated embodiments are examples of the present system and method and do not limit the scope thereof.

FIG. 1 is a side view of an assembled active compression orthopedic screw system, according to one exemplary embodiment.

FIG. 2 is a perspective exploded view illustrating the components of the active compression orthopedic bone screw system of the exemplary embodiment illustrated in FIG. 1.

FIGS. 3A-3C illustrate a side, a perspective, and a bottom view, respectively, of a top screw portion of the exemplary active compression orthopedic screw system illustrated in FIG. 1, according to various exemplary embodiments.

FIG. 4A is a side view of an elastic member components of the exemplary active compression orthopedic screw system of FIG. 1, according to one exemplary embodiment.

FIG. 4B is a stress/strain diagram illustrating the properties of a super-elastic wire, according to one exemplary embodiment.

FIGS. 5A and 5B are respectively a side and a perspective view of a bottom screw portion of the exemplary active compression orthopedic screw system of FIG. 1, according to one exemplary embodiment.

FIG. 6 is a flow chart illustrating a method for inserting and compressively loading the exemplary compression orthopedic screw system of FIG. 1, according to one exemplary embodiment.

FIGS. 7A through 7D are various views of an assembled active compression orthopedic screw system being inserted into a plurality of bone segments, according to one exemplary embodiment.

FIG. 8 is a side view illustrating a Herbert type active compression screw system, according to one exemplary embodiment.

FIG. 9 is a perspective exploded view illustrating the components of the Herbert type active compression orthopedic bone screw system of the exemplary embodiment illustrated in FIG. 8.

FIGS. 100A-10C illustrate a side, a perspective, and a bottom view, respectively, of a top screw portion of the exemplary Herbert type active compression orthopedic screw system illustrated in FIG. 8, according to various exemplary embodiments.

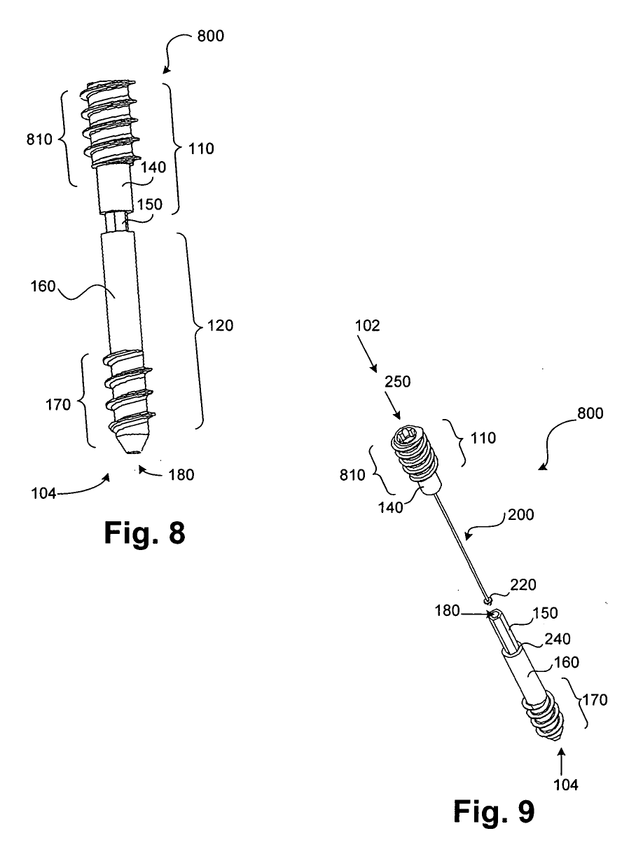

FIG. 11 illustrates a side view of an elastic member component of the exemplary active compression orthopedic screw system of FIG. 8, according to one exemplary embodiment.

FIGS. 12A and 12B are respectively a side and a perspective view of a bottom screw portion of the exemplary Herbert type active compression orthopedic screw system of FIG. 8, according to one exemplary embodiment.

FIGS. 13A through 13D are various views of an assembled Herbert type active compression orthopedic screw system being inserted into a plurality of bone segments, according to one exemplary embodiment.

FIG. 14 is a flow chart illustrating a method for inserting a pre-loaded active compression orthopedic screw system, according to one exemplary embodiment.

FIGS. 15A and 15B illustrate a side and a partial exploded view, respectively, of a pre-loadable active compression orthopedic screw system, according to one exemplary embodiment.

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings. Throughout the drawings, identical reference numbers designate similar but not necessarily identical elements.

### DETAILED DESCRIPTION

The present specification describes a system and a method for providing an actively compressing screw system that compresses secured bone segments. Particularly, according to one exemplary embodiment, the present specification describes the structure of an orthopedic bone system that can be pre-loaded prior to insertion or effectively loaded during insertion into a desired orthopedic site to post-operatively provide active compression across a facture. According to one exemplary embodiment, the exemplary actively compressing screw system includes a top screw portion slideably coupled to a bottom screw portion. Further, the top screw portion and the bottom screw portion are coupled by an elastic member configured to be tensioned and provide active compression between the top and bottom screw portions. Further details of the present exemplary system and method will be provided below.

The present exemplary active compression orthopedic screw system will be described herein, for ease of explanation only, in the context of a bone screw assembly configured to stabilize facet joints or odontoid fractures of the spine and block movement while fusion occurs. However, the methods and structures disclosed herein are intended for application in any of a wide variety of bones and fractures, as will be apparent to those of skill in the art in view of the disclosure herein. For example, the bone fixation device of the present exemplary system and method is applicable in a wide variety of fractures and osteotomies in the hand, such as interphalangeal and metacarpophalangeal arthrodesis, transverse phalangeal and metacarpal fracture fixation, spiral phalangeal and metacarpal fracture fixation, oblique phalangeal and metacarpal fracture fixation, intercondylar phalangeal and metacarpal fracture fixation, phalangeal and metacarpal osteotomy fixation as well as others known in the art. A wide variety of phalangeal and metatarsal osteotomies and fractures of the foot may also be stabilized using the bone fixation device of the present exemplary system and method. These include, among others, distal metaphyseal osteotomies such as those described by Austin and Reverdin-Laird, base wedge osteotomies, oblique diaphyseal, digital arthrodesis as well as a wide variety of others that will be known to those of skill in the art. Fractures of the fibular and tibial malleoli, pilon fractures and other fractures of the bones of the leg may also be fixated and stabilized with the present exemplary system and method. Each of the foregoing may be treated in accordance with the present system and method, by advancing one of the active compression screw systems disclosed herein through a first bone component, across the fracture, and into the second bone component to fix the fracture.

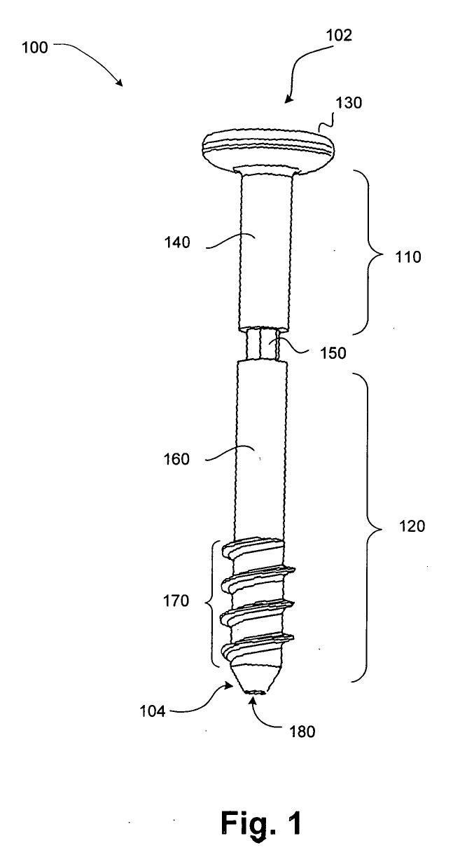

According to another exemplary embodiment, the active compression screw system of the present exemplary system and method may also be used to attach tissue or structure to the bone, such as in ligament reattachment and other soft tissue attachment procedures. The fixation device may also be used to attach sutures to the bone, such as in any of a variety of tissue suspension procedures. For example, according to one exemplary embodiment, soft tissue such as capsule, tendon, or ligament may be affixed to bone. It may also be used to attach a synthetic material such as marlex mesh, to bone or allograft material such as tensor fascia lata, to bone. In the process of doing so, retention of the material to bone may be accomplished with an enlarged head portion of the active compression orthopedic screw system shown in FIG. 1 to accept a suture or other material for facilitation of this attachment. The ability of the present active compression orthopedic screw prevents loosening of the screw, thereby reducing the likelihood that the attached tissue or structure will be prematurely released from the bone.

As mentioned previously, traditional bone fixation screw systems and other bone fixation devices are designed to limit motion within the coupled bone segments or other fused masses. However, a German doctor by the name of Julius Wolff demonstrated that bone grows when in compression and resorbs in the absence thereof. In other words, the form of a bone being given, the bone elements place or displace themselves in the direction of functional pressure. Consequently, the present exemplary system and method provides an orthopedic screw system configured to provide a post-operative “active” compressive force on the joined bone segments or fusion mass. As used herein, the term “active” shall be interpreted as referring to a screw system configured to provide a compressive force; rather than a “passive” fastener which would allow a compressive force but not itself provide a compressive force.

In the following description, certain specific details are set forth in order to provide a thorough understanding of various embodiments of the present active compression orthopedic screw system and method. However, one skilled in the relevant art will recognize that the present exemplary system and method may be practiced without one or more of these specific details, or with other methods, components, materials, etc. In other instances, well-known structures associated with orthopedic screw systems have not been shown or described in detail to avoid unnecessarily obscuring descriptions of the present exemplary embodiments.

As used in the present specification, and in the appended claims, the term “wire” shall be interpreted to include any number of members having a square, round, or oblong cross-section, configured to store energy. Specifically, a wire, when used in the present specification or the appended claims, includes any ligament whether a single member or a plurality of intertwined ligaments.

Further, as used herein, the term “slideably coupled” shall be interpreted broadly as including any coupling configuration that allows for relative translation between two members, wherein the translation may be linear, non-linear, or rotational.

Unless the context requires otherwise, throughout the specification and claims which follow, the word “comprise” and variations thereof, such as, “comprises” and “comprising” are to be construed in an open, inclusive sense, that is as “including, but not limited to.”

Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment. Furthermore, the particular features, structures, or characteristics may be combined in any suitable manner in one or more embodiments.

Exemplary Structure

FIG. 1 illustrates an assembled active compression orthopedic screw system (100), according to one exemplary embodiment. As illustrated, the exemplary active compression orthopedic screw system (100) includes a number of components including, but in no way limited to, a top screw portion (110) and a bottom screw portion (120) slideably coupled by an engagement member (150).

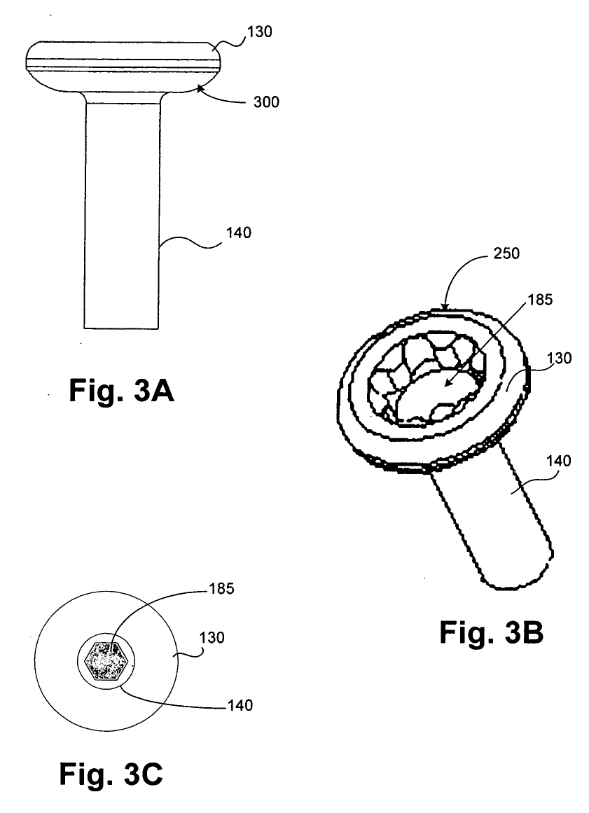

According to the exemplary embodiment illustrated in FIG. 1, the top screw portion (110) is disposed on the proximal end (102) of the active compression screw system (100) and includes a number of components including, but in no way limited to, a head portion (130) and an upper shaft portion (140) protruding from the head portion. Further, the top screw portion (110) includes a shaft reception orifice (185; FIGS. 3B and 3C) configured to slideably engage the engagement member (150) formed on the distal end of the bottom screw portion (120).

The bottom screw portion (120) of the active compression screw system (100) includes a lower shaft (160) having a lower thread portion (170) formed thereon. Additionally, an inner channel (180) is concentrically formed in the lower shaft (160), according to one exemplary embodiment. As shown, the engagement member (150) is formed on the proximal end of the bottom screw portion (120) to slideably engage the top screw portion (110).

While the present exemplary embodiment includes the engagement member (150) formed on the distal end of the bottom screw portion (120) and a corresponding shaft reception orifice (185; FIGS. 3B and 3C), the engagement member (150) may alternatively be formed on the proximal end of the top screw portion (110) and a corresponding shaft reception orifice (185; FIGS. 3B and 3C) formed in the bottom screw portion (120). Further, any number of slideable or rotationally translating coupling configurations may be incorporated to couple the top screw portion (110) and the bottom screw portion (120).

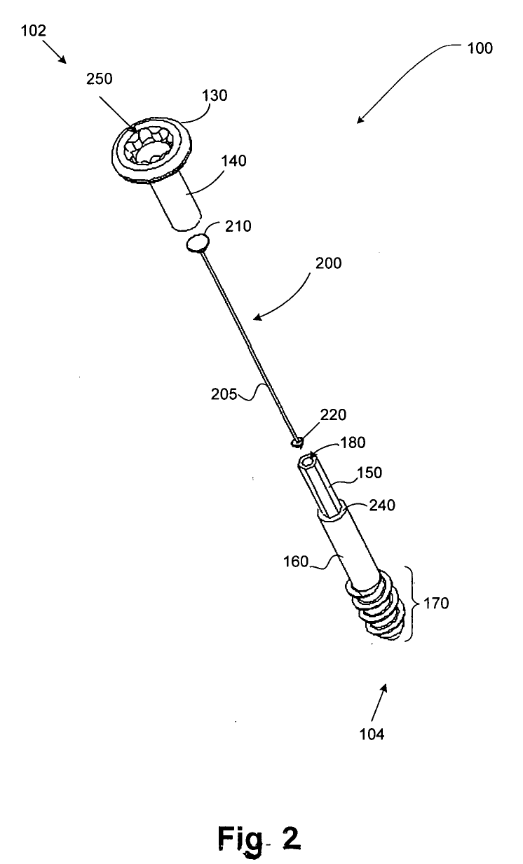

FIG. 2 is an exploded view further illustrating the components of the exemplary active compression screw system (100), according to one embodiment. As shown, an elastic member (200) having a proximal retention member (210) and a distal retention member (220) disposed on each end of an elastic wire (205) is positioned within the upper shaft (140) and the lower shaft (160). According to one exemplary embodiment, described in further detail below, the proximal retention member (210) and the distal retention member (220) securely couple the proximal end of the elastic member (200) to the top screw portion (110) and the bottom screw portion (120) respectively. Once coupled, relative separation of the top screw portion (110) from the bottom screw portion (120) introduces tension in the elastic member (200), thereby compressively loading it. Further details of each component of the exemplary active compression screw system (100) shown in FIGS. 1 and 2 will be provided below with reference to FIGS. 3A through 5B.

FIGS. 3A through 3C illustrate various views of the top screw portion (110) of the active compression screw system, according to one exemplary embodiment. As shown, in FIG. 3A, the exemplary top screw portion (110) includes a generally planar head (130) having a substantially smooth under surface (300). A substantially cylindrical upper shaft (140) is coupled to the smooth under surface (300). According to the present exemplary embodiment, the generally planar head (130) is used since a screw with a head is known to generate more compression across a fracture than a screw embodiment without a head. Further, the generally planar head (130) may provide a site for connection of a tissue or other structure to a desired bone segment. Alternatively, a top screw portion without the inclusion of a generally planar head may be used, as will be described below with reference to FIGS. 8 through 13D.

Continuing with FIGS. 3A through 3C, a driving feature (250) is formed on the proximal surface of the head (130). As shown, the driving feature (250) is a multi-toothed female reception orifice configured to receive a mating driver. A female reception orifice can be used to reduce the profile of the head (130). Any number of driving feature (250) configurations may be used including, but in no way limited to, a Phillips head configuration, an Allen head configuration, and the like. Alternatively, a male driving feature (250) may be used.

FIG. 3B also illustrates a shaft reception orifice (185) formed in the center of the top screw portion (110; FIG. 1). As illustrated in FIG. 3C, the distal portion of the shaft reception orifice (185) is sized and shaped to slideably receive the engagement shaft (150; FIG. 2) of the bottom screw portion (120; FIG. 1). According to one exemplary embodiment, the shaft reception orifice (185) has an upper diameter that is less than the largest diameter of the proximal retention member (210) of the elastic member (200). Consequently, interference may exist between the proximal retention member (210) and the top screw portion (110; FIG. 1).

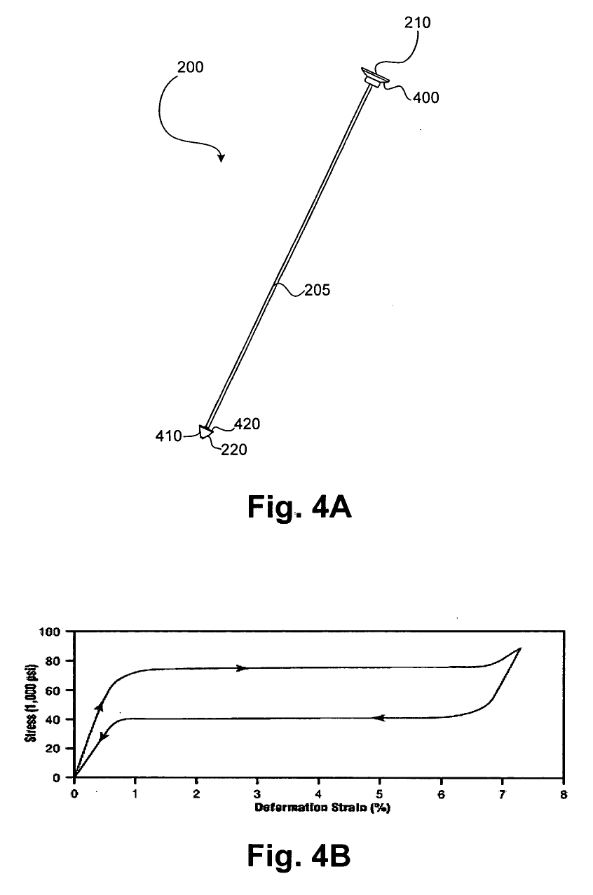

FIG. 4A illustrates the elastic member (200), according to one exemplary embodiment. As shown, the exemplary elastic member (200) includes a proximal retention member (210) and a distal retention member disposed on opposite ends of an elastic wire (205). As shown, the exemplary proximal retention member (210) includes an interference face (400) configured to interfere with a feature of the top screw portion (110) when assembled. Similarly, the exemplary distal retention member (220) is defined by an inclined face (410) dropping off to form a retraction stop (420). The exemplary distal retention member (220) is configured to be fixedly retained in the bottom screw portion (120; FIG. 1). While exemplary configurations of the proximal (210) and distal retention members (220) are illustrated herein, any retention means for fixedly coupling the elastic wire (205) to the top screw portion (110; FIG. 1) and the bottom screw portion (120; FIG. 1) may be used.

The elastic wire (205) illustrated in FIG. 4A may be a super-elastic member configured to provide a compressive force to the present exemplary active compression orthopedic screw system (100). According to one exemplary embodiment, the elastic wire (205) is concentrically placed within the body of the active compression screw system (100). According to the exemplary embodiment illustrated in FIG. 2, a lumen is formed in the center of the screw system (100) to allow placement of the elastic wire (205) therein.

According to the exemplary embodiment illustrated in FIG. 2, the elastic wire (205) is disposed within the active compression screw system (100). However, the elastic wire (205) may be disposed in or around any portion of the exemplary screw system (100), compressibly coupling the top (110) and bottom (120) screw portions. Alternatively, any number of elastic wires (205) may be used to provide an active compression force on the exemplary orthopedic screw system (100).

According to one exemplary embodiment in which the elastic member (200) is disposed within the active compression screw system (100), the retention members (210, 220) may be coupled to each end of the elastic wire (205) after the elastic wire is coupled to the screw system. While the exemplary elastic wire (205) may be formed of any number of elastic materials, the present exemplary wire member is made, according to one exemplary embodiment, of a super-elastic material.

The super-elastic material used to form the exemplary elastic wire (205) may be a shape memory alloy (SMA), according to one exemplary embodiment. Super-elasticity is a unique property of SMA. If the SMA is deformed at a temperature slightly above its transition temperature, it quickly returns to its original shape. This super-elastic effect is caused by the stress-induced formation of some martensite above its normal temperature. Because it has been formed above its normal temperature, the martensite reverts immediately to undeformed austenite as soon as the stress is removed. FIG. 4B is a stress/strain diagram illustrating the properties of a super-elastic material used for the exemplary elastic wire (205), according to one exemplary embodiment. As shown, an initial increase in deformation strain creates great stresses in the material, followed by a stress plateau with the continued introduction of strain. As the strain is reduced, the stress again plateaus, providing a substantially constant level of stress. This property of the super-elastic material allows the exemplary elastic wire (205) to be preloaded with compressive forces prior to or once inserted in desired bone segments.

According to one exemplary embodiment, the super-elastic material used to form the elastic wire (205) includes, but is in no way limited to a shape memory alloy of nickel and titanium commonly referred to as nitinol. The elastic wire (205) may be formed of nitinol, according to one exemplary embodiment, because nitinol wire provides a low constant force at human body temperature. The transition temperature of nitinol wires are made so that they generate force at the temperature of about 37° C. (98.6° F.). Additionally, nitinol exhibits a reduction in elongation at a rate of approximately 10%, which is approximately equal to the subsidence rate of an orthopedic body.

According to one exemplary embodiment, the diameter of the elastic wire (205) may be selectively chosen to provide a desired compressive force. According to one exemplary embodiment, the greater the diameter of the elastic wire (205), the greater the compressive force will be provided, given a constant separation length. Consequently, a surgeon may selectively choose a diameter of the elastic wire to suit a particular procedure.

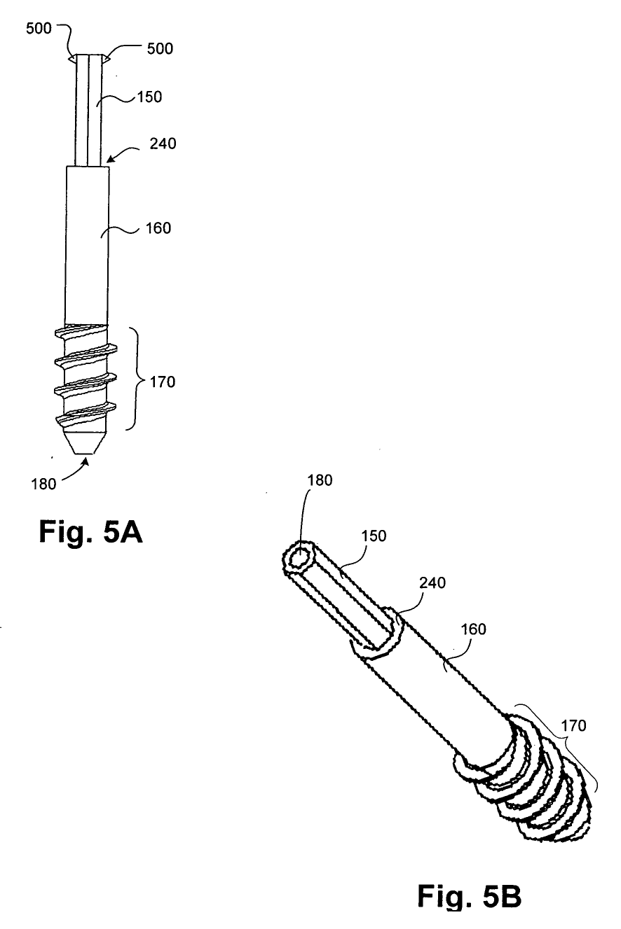

Continuing with the components of the exemplary active compression screw system (100; FIG. 1), FIGS. 5A and 5B show various views of a bottom screw portion (120) of the present exemplary screw system. As shown, the bottom screw portion (120) includes an engagement member (150) protruding from a lower shaft portion (160). While the exemplary engagement member (150) illustrated in FIGS. 5A and 5B is shown as having a substantially hexagonal cross-sectional profile, the engagement member (150) may assume any number of cross-sectional shapes.

Additionally, as illustrated in FIG. 5A, one or more stop member(s) (500) can be formed on the engagement member (150). According to this exemplary embodiment, the one or more stop member(s) (500) may be configured to interact with a protrusion (not shown) in the shaft reception orifice (185; FIGS. 3B and 3C). The placement of the stop member(s) (500) on the engagement member (150) allows for the slideable translation of the engagement member within the shaft reception orifice during use, while capturing the elastic member (200) in case of fatigue failure. Specifically, should the elastic member (200) fail, interference between the protrusion (not shown) in the shaft reception orifice (185; FIGS. 3B and 3C) and the one or more stop member(s) (500) will prevent the top screw portion (110; FIG. 1) from completely separating from the bottom screw portion (120; FIG. 1) and will cause the elastic member (200) to be retained within the exemplary active compression screw (100; FIG. 1).

Additionally, selective placement of the one or more stop members (500) on the engagement member (150) can vary the degree of subsidence permitted by the exemplary screw system (100). Specifically, placement of the one or more stop members (500) defines the maximum relative separation between the top screw portion (110; FIG. 1) and the bottom screw portion (120; FIG. 1).

At the interface between the lower shaft portion (160) and the engagement member (150), the varying diameters defines an engagement stop (240) that limits the slideable position of the top screw portion (110; FIG. 1) relative to the bottom screw portion (120; FIG. 1). Additionally, a lower thread portion (170) is formed on the lower part of the lower shaft portion (160). According to one exemplary embodiment, the lower thread portion (170) may include a self-tapping leading edge to provide the present exemplary screw system with the ability to remove bone material as it is being inserted into bone segment(s), eliminating a step of a surgeon drilling a pilot hole prior to insertion of the screw. While a threaded portion is illustrated as providing a means for coupling the bottom screw portion (120) to a desired bone segment, any number of fixation means may be used to fix the bottom screw portion including, but in no way limited to, adhesives, expandable walls, and the like.

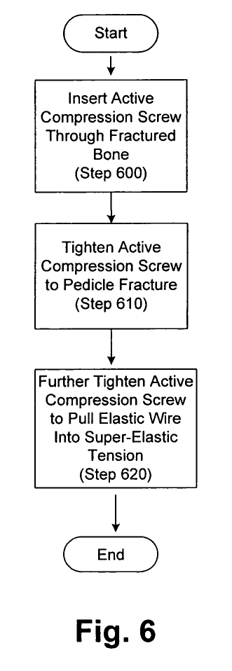

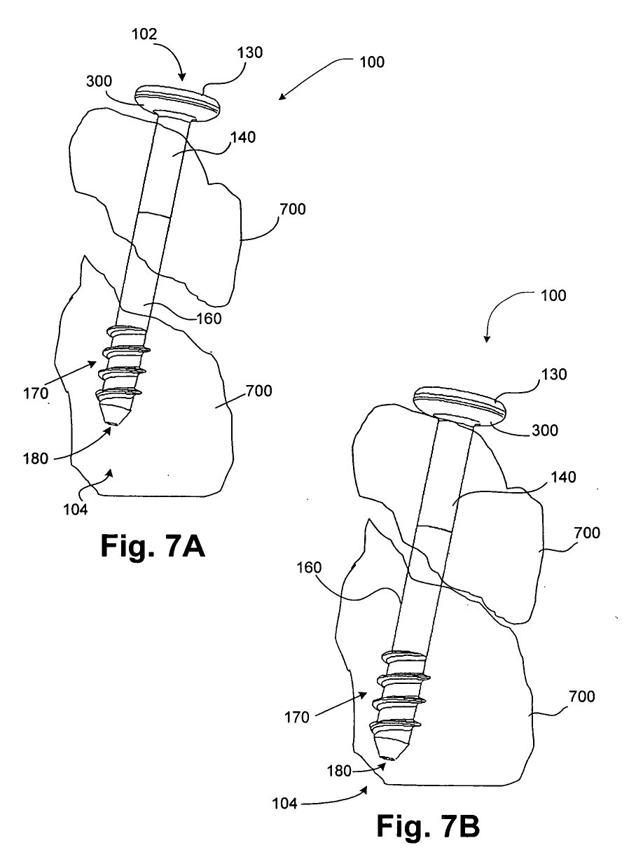

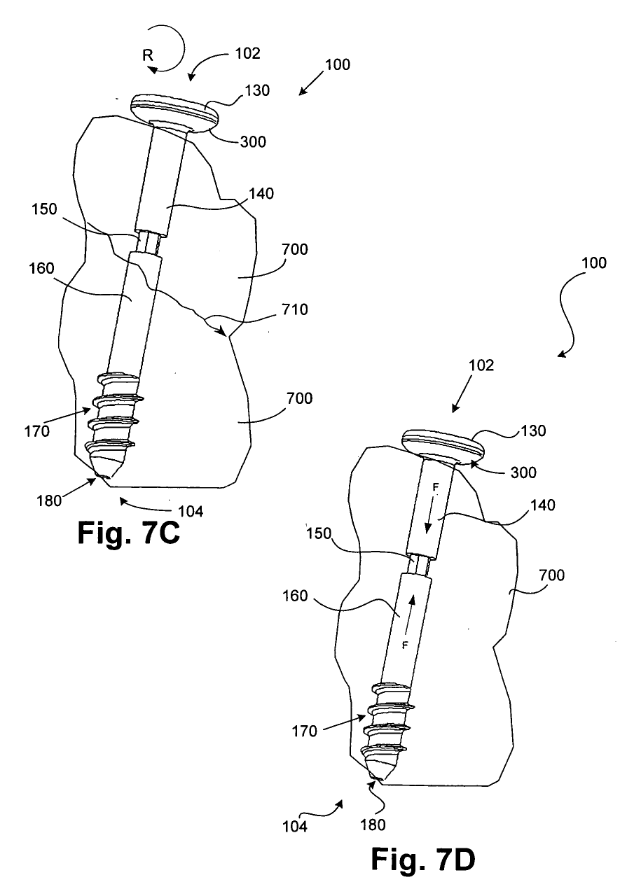

Additionally, FIG. 5B illustrates the inner channel (180) formed in the bottom screw portion (120; FIG. 1) of the present exemplary active compression screw system (100; FIG. 1). According to one exemplary embodiment, the inner channel (180) formed in the exemplary bottom screw portion may include one or more protrusions configured to provide an interference with the distal retention member (220; FIG. 4A) when assembled. Further detail of the function and operation of the exemplary active compression orthopedic screw system (100) will be described below with reference to FIGS. 6-7D.

Exemplary Method

FIG. 6 illustrates an exemplary method for installing the active compression orthopedic screw system (100; FIG. 1), according to one exemplary embodiment. As illustrated in FIG. 6, the present exemplary method for installing the active compression orthopedic screw system (100; FIG. 1) includes inserting the active compression screw through a fractured bone (step 600), tightening the active compression screw to reduce the fracture (step 610), and then further tightening the active compression screw to pull elastic wire into super-elastic tension (step 620). When maintained in the fractured bone, the present exemplary active compression orthopedic screw system post-operatively applies compression across the fracture, thereby promoting bone growth. Further details of each step of the present exemplary method will be provided below with reference to FIGS. 7A through 7D.

As illustrated in FIG. 7A, the first step of the exemplary method is to insert the exemplary active compression screw assembly through a plurality of bone segments (step 600). According to one exemplary embodiment, the present active compression orthopedic screw system (100; FIG. 1) can be assembled prior to implantation or in-situ. FIGS. 7A through 7D illustrate an assembled orthopedic screw system, according to one exemplary embodiment. As shown in FIG. 7A, the assembled screw system in its un-disturbed state includes the top screw portion (110) immediately adjacent to the bottom screw portion (120). In this exemplary state, the strains introduced on the elastic member (200; FIG. 2) are minimized. Further, when assembled, the engagement member (150; FIG. 5A) is disposed within the shaft reception orifice (185; FIG. 3C) of the top screw portion (110). Additionally, the proximal retention member (210; FIG. 2) and the distal retention member (220; FIG. 2) are independently coupled to the top screw portion (110) and the bottom screw portion (120) respectively by any number of mechanisms including, but in no way limited to, adhesives, mechanical fasteners, and/or an interference fit.

Once assembled, as illustrated in FIG. 7A, the exemplary active compression screw system (100) can be inserted through a plurality of bone segments (700). As shown, the exemplary active compression screw system (100) may be selectively placed in each of the multiple bone segments (700) being joined, in order to optimize the alignment of the fracture interfaces. Insertion of the active compression screw system (100) may be performed either by pre-drilling a pilot hole in the bone segments (700) or, alternatively, allowing a self-tapping thread of the lower thread portion (170) to remove the interfering bone mass. Regardless of the method of inserting the exemplary active compression screw system (100), once inserted, the screw is then tightened, drawing the bone segments together (step 610).

As illustrated in FIG. 7B, tightening of the exemplary active compression screw system (100) causes the bone segments (700) to be drawn together, mating the fracture interfaces. Consequently, the fracture is reduced. However, as shown in FIG. 7B, the elastic member (200; FIG. 2) is not stressed, resulting in little to no active compression. Consequently, the screw is further tightened, pulling the elastic member (200; FIG. 2) into super-elastic tension (step 620).

FIG. 7C illustrates the present exemplary active compression screw system (100) in super-elastic tension, according to one exemplary embodiment. As shown, continued rotation (R) of the active compression screw system (100) after the bone segments (700) have been fully reduced continues to drive the bottom screw portion (120) into the lower bone segment (700), as indicated by the arrow in FIG. 7C. However, the head (130) portion of the screw assembly prevents the top screw portion (110) from continuing into the bone segment (700). Rather, the smooth undersurface (300) of the head portion (130) rotates on the surface of the bone segment (700). Consequently, a relative translation of the bottom screw portion (120) away from the top screw portion (110) occurs. As the respective screw portions are separated, the portions continue to be coupled, and consequently translate any rotational force (R), via the engagement member (150). As mentioned previously, the elastic member (200; FIG. 2) is independently coupled to each of the top screw portion (110) and the bottom screw portion (120). Consequently, the relative translation of the bottom screw portion away from the top screw portion introduces a super-elastic strain into the elastic member (200; FIG. 2), placing the active compression orthopedic screw system (100) in a distracted state.

As illustrated in FIG. 7D, distracting the present exemplary active compression orthopedic screw system (100) causes the super-elastic tension of the elastic or super-elastic wire (205) to continuously apply active compression (F) across the fracture (710), thereby promoting bone growth and healing.

### Alternative Embodiments

While the above-mentioned exemplary active compression screw system (100) has been described in the context of a top screw portion (110; FIG. 1) having a substantially planar head (130) portion, any number of head configurations may be used to form the top screw portion (110), according to various embodiments. Specifically, FIGS. 8 and 9 illustrate a side and exploded perspective view, respectively, of a Herbert type active compression screw system (800). As illustrated in FIGS. 8 and 9, the top screw portion (110) may include an upper thread portion disposed on the upper shaft (140). According to the exemplary embodiment illustrated in FIGS. 8 and 9, a Herbert type active compression screw system may be used to reduce the likelihood of tissue irritation. Particularly, screw systems with a head (130; FIG. 1) are left proud of the surface of a bone segment when installed. Consequently, the head portion may cause irritation to the surrounding tissue. In contrast, a Herbert type active compression screw system (800), as illustrated in FIGS. 8 and 9, has no head and sits entirely within the bone, greatly reducing the likelihood of tissue irritation.

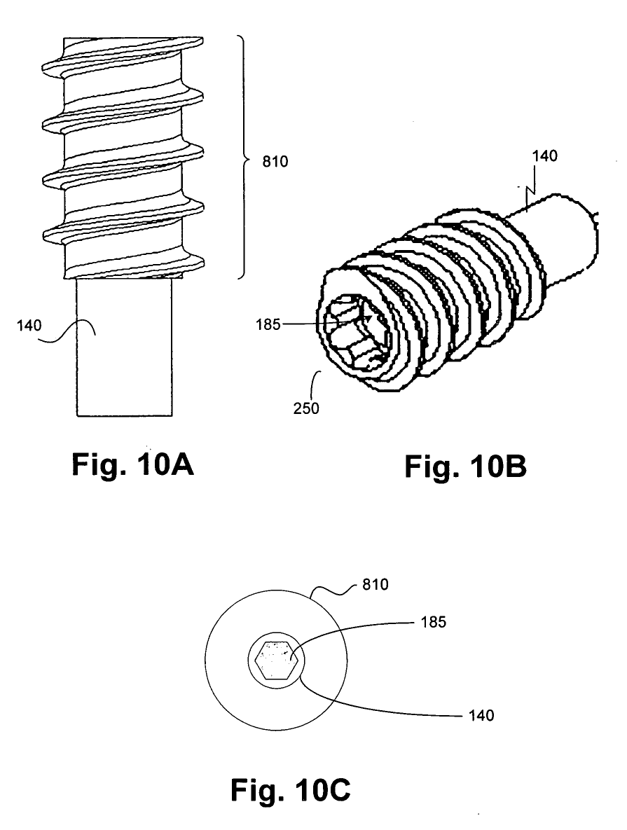

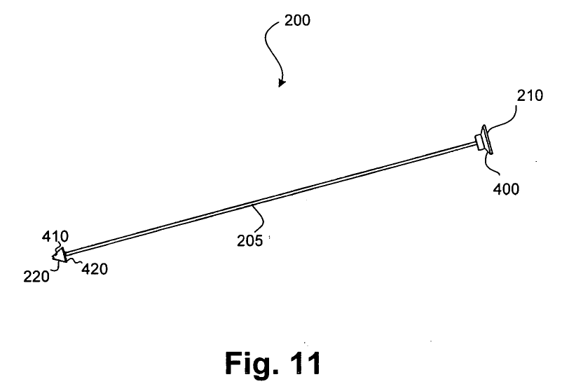

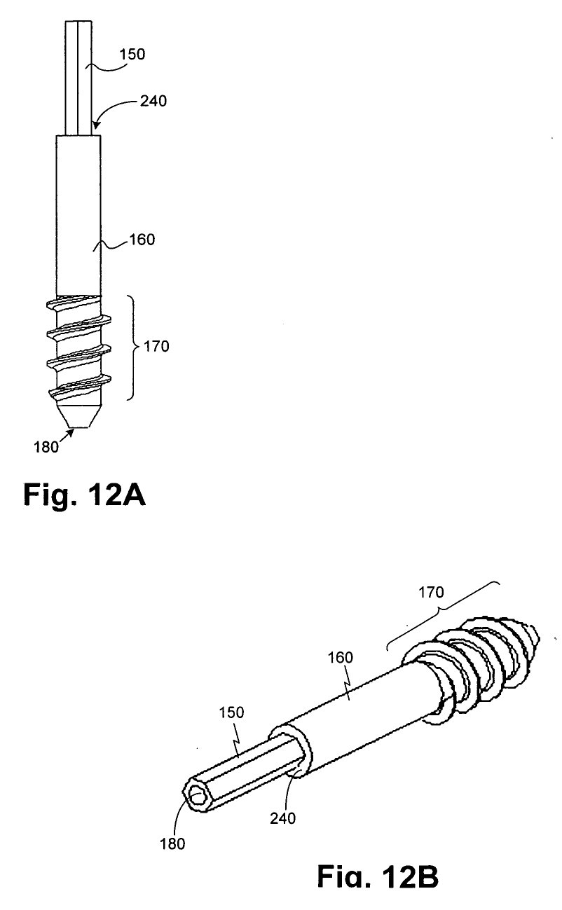

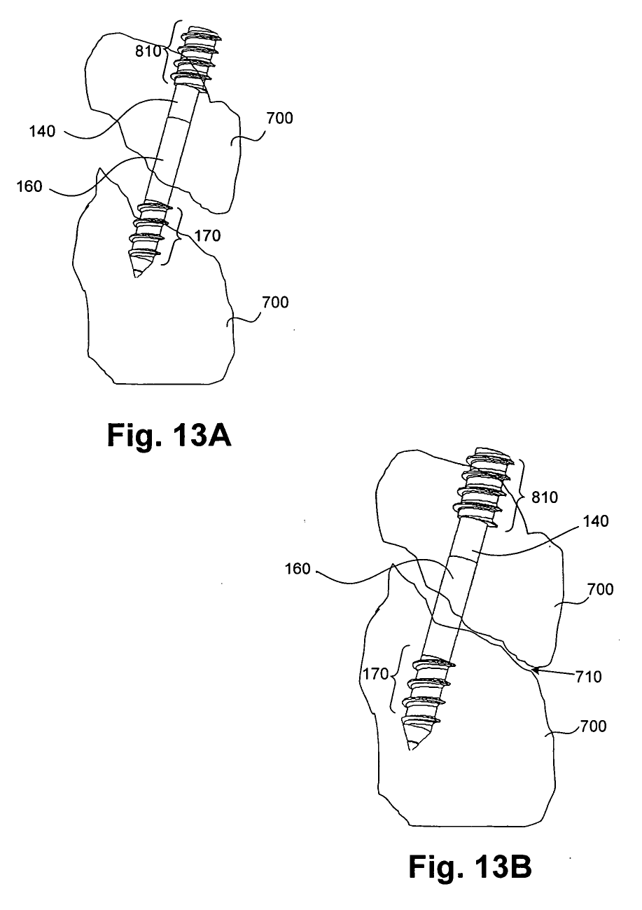

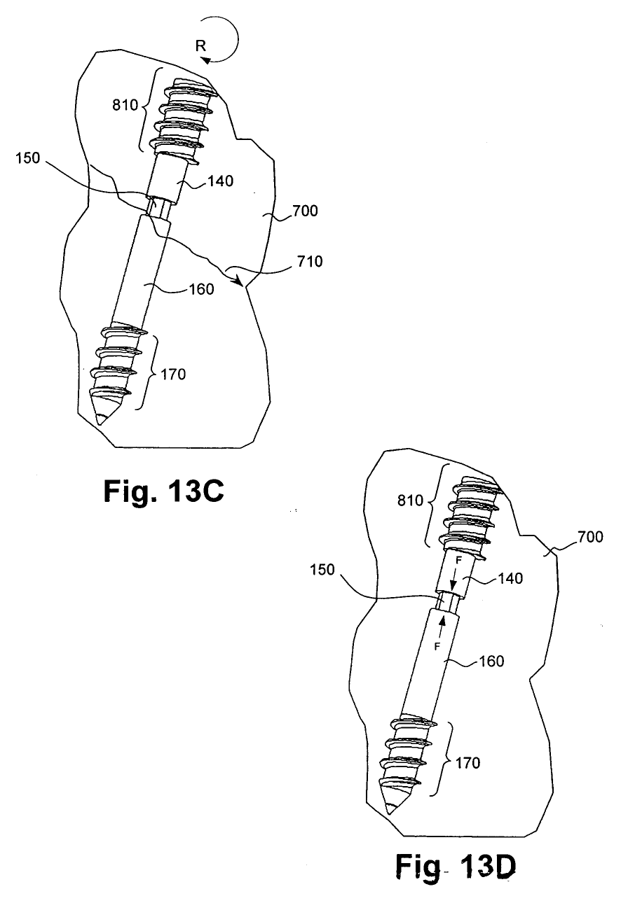

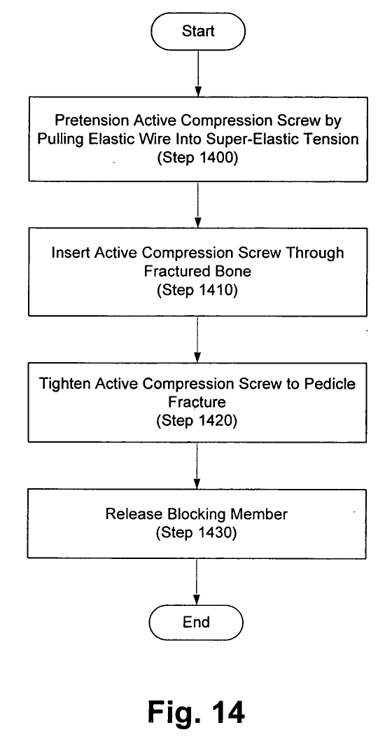

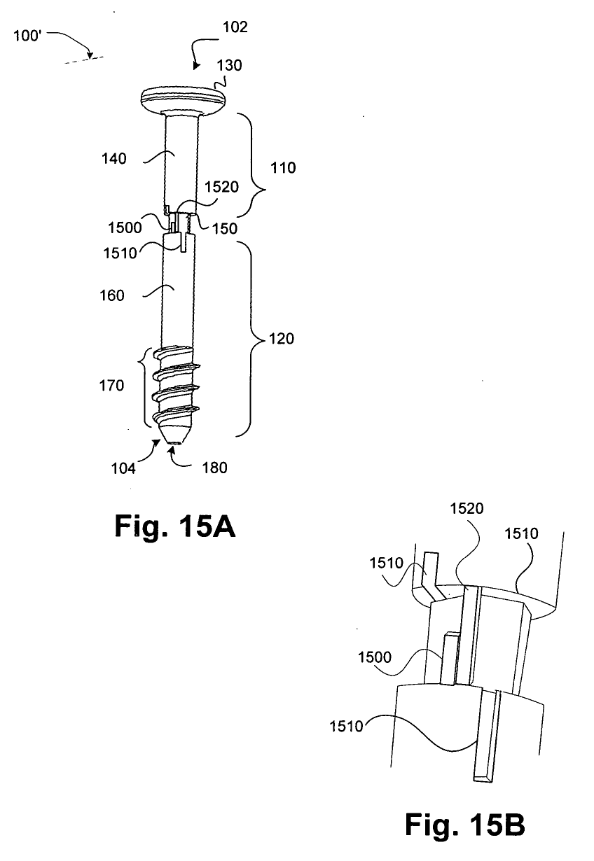

As shown in FIGS. 10A through 12B, the exemplary Herbert type active compression screw system (800) includes similar components as the exemplary active compression screw system (100; FIG. 1) illustrated in FIG. 1, with the notable exception of the top screw portion (110). According to the exemplary embodiment illustrated in FIGS. 10A through 10C, the upper thread portion (810) of the top screw portion (110) includes a number of tapered threads. According to one embodiment, the pitch of the threads formed on the upper thread portion (810) differ from the pitch of the threads formed on the lower thread portion (170; FIG. 12A). Specifically, according to one exemplary embodiment, the threads formed on the upper thread portion (810) of the exemplary Herbert type active compression screw system (800) have a shallower pitch than the threads formed on the lower thread portion (170). Consequently, when the top screw portion (110) and the bottom screw portion (120) are driven into a similar material by the same rotational force and velocity, the lower threaded portion (170) will cause the bottom screw portion (120) to be driven faster than the top screw portion (110), resulting in separation of the two.

FIGS. 13A through 13D illustrate an insertion of the present exemplary Herbert type active compression screw system (800) into a plurality of bone segments (700) using the method of FIG. 6. As illustrated in FIG. 13A, once assembled, the Herbert type active compression screw system (800) can be inserted into the bone segments (step 600; FIG. 6). Initially, only the lower thread portion (170) of the bottom screw portion (120) is driven into the bone segments (700) and no differential exists between the top and bottom screw portions. As the screw is tightened (step 610; FIG. 6), the bone segments (700) are drawn together, thus reducing the fracture (710). Once the fracture (710) is fully reduced, as shown in FIG. 13C, further tightening of the Herbert type active compression screw system (800) causes the top screw portion (110) and the bottom screw portion (120) to be driven at differing translational rates. Consequently, the elastic member (200) is pulled into super-elastic tension, as shown in FIG. 13D. Similar to the exemplary embodiment illustrated above, distracting the exemplary Herbert type active compression orthopedic screw system (100) causes the super-elastic tension of the elastic or super-elastic wire (205) to continuously apply active compression (F) across the fracture (710), thereby promoting bone growth and healing.

While the above-mentioned systems and methods may be used for normal bones, the exemplary method illustrated in FIG. 14 allows for the insertion of an active compression screw in an osteoporotic bone. As illustrated in FIG. 14, the exemplary method for osteoporotic bone begins by first pre-tensioning an active compression screw by pulling the elastic wire into super-elastic tension (step 1400). According to the present exemplary method, an osteoporotic bone may not be sufficiently strong to withstand the high forces needed to pre-load the elastic or super-elastic wire to desired levels. Consequently, the exemplary method illustrated in FIG. 14 allows for pre-tensioning of the active compression screw.

When the active compression screw is pre-tensioned, it may then be inserted into the osteoporotic bone segments (step 1410) and tightened (step 1420). During the insertion and tightening of the active compression screw in the osteoportoic bone segments, the active compression screw is maintained in its pre-tensioned state. Accordinglty, any number of systems may be used to maintain the desired levels of tension in the elastic or super-elastic wire during insertion of the active compression screw. FIGS. 15A and 15B illustrate just one exemplary system for maintaining the desired levels of tension during insertion.

As shown in FIGS. 15A and 15B, a blocking member (1520) is formed on the top screw portion (110). As shown, the blocking member (1520) is configured to maintain the active compression screw (100″) in an expanded state. The active compression screw (100′) may be driven clockwise to drive the active compression screw into the osteoporotic bone segments. As illustrated, driving the top screw portion (110) will force the blocking member (1520) into a rotation stop (1500) disposed on the bottom screw portion (120). Once the blocking member is engaged with the rotation stop (1500), rotational force imparted on the top screw portion (110) will be translated to the bottom screw portion (120).

Once the active compression screw (100′) is sufficiently driven, the blocking member can be released (step 1430; FIG. 14), allowing the active compression screw to impart an active compressive force on the osteoporotic bone segments. According to the exemplary embodiment illustrated in FIGS. 15A and 15B, the blocking member (1520) may be released by rotating the top screw portion (110) counter-clockwise. When driven counter-clockwise, the blocking member (1520) and the rotation stop (1500) are aligned with corresponding recesses (1510) formed in each of the upper shaft (140) and the lower shaft (160). The recesses (1510) are sized to receive the blocking member (1520) and the rotation stop (1500), allowing the active compression screw to impart an active compressive force on the osteoporotic bone segments.

In conclusion, the present exemplary systems and methods provide for an active compression orthopedic screw system. Particularly, the present exemplary system is configured to actively impart a compressive force on a plurality of bone segments, thereby promoting bone growth. Consequently, the present exemplary active compression orthopedic screw system increases osteogenic stimulation as well segment stabilization.

The preceding description has been presented only to illustrate and describe the present method and system. It is not intended to be exhaustive or to limit the present system and method to any precise form disclosed. Many modifications and variations are possible in light of the above teaching.

The foregoing embodiments were chosen and described in order to illustrate principles of the system and method as well as some practical applications. The preceding description enables others skilled in the art to utilize the method and system in various embodiments and with various modifications as are suited to the particular use contemplated. It is intended that the scope of the present exemplary system and method be defined by the following claims.

What is claimed is:

1. An active compression orthopedic screw, comprising:
a first shaft member positioned at a distal end of said screw;
a second shaft member positioned at a proximal end of said screw; and
an elastic member having a first and a second end;
wherein said first end of said elastic member is coupled to said first shaft member and said second end of said elastic member is coupled to said second shaft member, said elastic member being configured to exert a force drawing said first and second shaft members together.

2. The screw of claim 1, wherein said first shaft member and said second shaft member are slideably coupled.

3. The screw of claim 2, further comprising:
a protrusion extending from one of said first or second shaft member; and
a protrusion receiving orifice formed in one of said first or second shaft member not having said protrusion;
said protrusion receiving orifice being configured to slideably receive said protrusion.

4. The screw of claim 1, further comprising threads disposed on an outer surface of said first shaft member.

5. The screw of claim 4, wherein said threads comprise self-tapping threads.

6. The screw of claim 4, wherein said second shaft member comprises a head disposed on a proximal end of said second shaft member.

7. The screw of claim 4, further comprising threads disposed on an outer surface of said second shaft member;
wherein said threads disposed on said outer surface of said first shaft member have a first pitch;
wherein said threads disposed on said outer surface of said second shaft member have a second pitch; and
wherein said first pitch and said second pitch are not equal.

8. The screw of claim 1, wherein said elastic member comprises a shape memory alloy.

9. The screw of claim 8, wherein said shape memory alloy comprises Nitinol.

10. The screw of claim 1 wherein said elastic member is at least partially disposed in each of said first shaft member and said second shaft member.

11. The screw of claim 1, further comprising:
at least one blocking member protruding from said second shaft member;
a blocking member receiving recess formed in said first shaft member;
a rotation stop member protruding from said first member;
wherein said blocking member is configured to maintain a tension in said elastic member and engage said blocking member when said second shaft member is rotated in a first direction; and
wherein said blocking member is configured to release said tension in said elastic member and enter said blocking member receiving recess when said second shaft member is rotated in a second direction.

12. A system for coupling a tissue to a bone segment comprising:
a first shaft member positioned at a distal end of said screw;
a second shaft member positioned at a proximal end of said screw, said second shaft member including a tissue coupling protrusion; and
an elastic member having a first and a second end;
wherein said first end of said elastic member is coupled to said first shaft member and said second end of said elastic member is coupled to said second shaft member, said elastic member being configured to exert a force drawing said first and second shaft members together.

13. The coupling system of claim 12, wherein said tissue coupling protrusion comprises a head.

14. The coupling system of claim 12, wherein said first shaft member and said second shaft member are slideably coupled.

15. The coupling system of claim 14, further comprising:
a protrusion extending from one of said first or second shaft member; and
a protrusion receiving orifice formed in one of said first or second shaft member not having said protrusion;
said protrusion receiving orifice being configured to slideably receive said protrusion.

16. The coupling system of claim 12, wherein said elastic member comprises a shape memory alloy.

17. The coupling system of claim 16, wherein said shape memory alloy comprises Nitinol.

18. An active compression orthopedic screw, comprising:
a first shaft member positioned at a distal end of said screw including threads disposed on an outer surface of said first shaft member;
a second shaft member positioned at a proximal end of said screw;
a protrusion extending from one of said first or second shaft member; and
a protrusion receiving orifice formed in one of said first or second shaft member not having said protrusion;
said protrusion receiving orifice being configured to slideably receive said protrusion; and
a shape memory alloy elastic member having a first and a second end;
wherein said first end of said elastic member is coupled to said first shaft member and said second end of said elastic member is coupled to said second shaft member, said elastic member being configured to exert a force drawing said first and second shaft members together.

19. The screw of claim 18, wherein said shape memory alloy comprises Nitinol.

20. The screw of claim 18, wherein said second shaft member comprises a head disposed on a proximal end of said second shaft member.

21. The screw of claim 18, further comprising threads disposed on an outer surface of said second shaft member;
wherein said threads disposed on said outer surface of said first shaft member have a first pitch;
wherein said threads disposed on said outer surface of said second shaft member have a second pitch; and
wherein said first pitch and said second pitch are not equal.

22. A method of providing post-operative compression on a fracture comprising:
inserting an active compression screw through a plurality of bone segments defining said fracture;
tightening said active compression screw to reduce said fracture; and
tensioning said active compression screw to actively compress said fracture.

23. The method of claim 22, wherein tensioning said active compression screw comprises continuing to tighten said active compression screw after said fracture is fully reduced.

24. The method of claim 22, wherein said tensioning said active compression screw comprises pulling a super-elastic wire within said active compression screw into super-elastic tension.

25. A method for joining osteoporotic bone segments with an active compression screw, comprising:
pre-tensioning said active compression screw;
locking said active compression screw in a pre-tensioned state;
inserting said pre-tensioned active compression screw in said osteoporotic bone segments;
tightening said pre-tensioned active compression screw to reduce a fracture defined by said osteoporotic bone segments; and
un-locking said active compression screw to permit active compression of said osteoporotic bone segments.
